SEQUENCE LISTING (24 sequences)

SEQ ID NO: 1 - DNA, 18 bp, Artificial sequence
  primer
ggaaaacataataatgct

SEQ ID NO: 2 - DNA, 24 bp, Artificial sequence
  primer
ttcgcgcttgcttagatactccag

SEQ ID NO: 3 - DNA, 30 bp, Artificial sequence
  primer
gaccagttatctccggcttgtgcattgaaa

SEQ ID NO: 4 - DNA, 27 bp, Artificial sequence
  primer
tcgtccgcataacccttgagcttttgc

SEQ ID NO: 5 - DNA, 33 bp, Artificial sequence
  primer
agactaaactggctgacggaatttatgcctctt

SEQ ID NO: 6 - DNA, 35 bp, Artificial sequence
  primer
ttggtgattttgaacttttgctttgccacggaacg

SEQ ID NO: 7 - DNA, 45 bp, Artificial sequence
  primer
cttgatatcgctagctcgtatgttgtgtggaattgtgagcggata

SEQ ID NO: 8 - DNA, 39 bp, Artificial sequence
  primer
accaacgcgtaatcgccccatcatccagccagaaagtga

SEQ ID NO: 9 - DNA, 24 bp, Artificial sequence
  primer
tgggtttgtggtagatagtgggcg

SEQ ID NO: 10 - DNA, 24 bp, Artificial sequence
  primer
gcgcttttatcaatggcaaccctg

SEQ ID NO: 11 - DNA, 34 bp, Artificial sequence
  primer
gccaggatccctgaccgccacacaagttatccag

SEQ ID NO: 12 - DNA, 35 bp, Artificial sequence
  primer
acctgtcgacgatgcaactcgccctcatgccagat

SEQ ID NO: 13 - DNA, 35 bp, Artificial sequence
  primer
ggcaggatccttgattggcgtacatgcactcaagc

SEQ ID NO: 14 - DNA, 34 bp, Artificial sequence
  primer
atatgtcgacgcgtgatgatttgctgggtggtgc

SEQ ID NO: 15 - DNA, 27 bp, Artificial sequence
  primer
acagaattccaaggggtgttatgagcc

SEQ ID NO: 16 - DNA, 24 bp, Artificial sequence
  primer
gtgtcggggctggcttaactatgc

SEQ ID NO: 17 - DNA, 27 bp, Artificial sequence
  primer
gggtcaaaaatccgcaatggctgaaaa

SEQ ID NO: 18 - DNA, 27 bp, Artificial sequence
  primer
agcacgtcagcaatctcaaatggggtg

SEQ ID NO: 19 - DNA, 1315 bp, Methylophilus methylotrophus
  CDS (219)..(1247)
ggaaaacataataatgctcccagattcccttaacgtgtcatcctgctcaaggttgacacgattcaggccgaatttttaagccgaaatcactggcttgaattcatggcttcgccatttcactgatggtcgatttatgaaataattagaagttatggtttggattatatagcgtttgtataaacagcgcatttttatcgctaataaagaaggtaatcagcatggatgacaacaaaagcaaagcgctcgccgccgcactttcccaaattgaaaaacagttcggtaaaggctccattatgcgcatgggcgatgctgatatcggcgaagacctgcaagtggtttccaccggctcactgggcctggatatcgcactgggggtgggtggcttgccacgtggccgtattatcgaaatttatggccctgagtcttccggtaaaaccacattgaccttgtccgcgattgccgaaatgcaaaagttgggcggtgtcgcagcatttatcgatgctgagcatgctctggatccacagtacgcggccaagctgggcgtgaatgtgcctgaattactgatttcacagcctgacaccggggagcaagcgttggaaattgccgatatgctggtacgctccggctccgtggatatcgtggttgttgactcggtggctgccttgaccccacgtgccgaaattgaaggtgaaatgggtgacagccacatgggcttgcaggcacgcctgatgtcacaggcattgcgtaagctcactggtaacatcaagcgtaccaatacgctggtgatttttatcaaccagatccgtatgaagatcggtgtcatgttcggtaatcctgaaacgaccactggcggtaacgcgctcaagttttactcttctgtccgtctcgatatccgccgtaccggtgcgattaaaaaaggcgacgaggtgattggctctgagaccaaggtgaaggtcatcaagaacaaggttgcgccgccgttcaagcaggctgaattcgacatcatgtacggcgaaggtatttcccgtctgggcgaaatcattgagttgggtacaaatttgaaactggttgagaaatcaggtgcgtggtacagctacaacggtgaaaaaatcggccagggtaaagaaaacgctaaagagttcctgcgcgagaatccagcgattgcggcagaaattgaagccaagattcgcgacaactctaatgtgctggcagatagcatgactgcggccagaagtgaggacgattaagcagcttgcgtcagccgattgaaaagtcgctgcgccagcgtgcgctggagtatctaagcaagcgcgaa

SEQ ID NO: 20 - protein, 342 aa, Methylophilus methylotrophus
MDDNKSKALAAALSQIEKQFGKGSIMRMGDADIGEDLQVVSTGSLGLDIALGVGGLPRGRIIEIYGPESSGKTTLTLSAIAEMQKLGGVAAFIDAEHALDPQYAAKLGVNVPELLISQPDTGEQALEIADMLVRSGSVDIVVVDSVAALTPRAEIEGEMGDSHMGLQARLMSQALRKLTGNIKRTNTLVIFINQIRMKIGVMFGNPETTTGGNALKFYSSVRLDIRRTGAIKKGDEVIGSETKVKVIKNKVAPPFKQAEFDIMYGEGISRLGEIIELGTNLKLVEKSGAWYSYNGEKIGQGKENAKEFLRENPAIAAEIEAKIRDNSNVLADSMTAARSEDD

SEQ ID NO: 21 - DNA, 2158 bp, Methylophilus methylotrophus
  CDS (514)..(1407)
gccaggatccctgaccgccacacaagttatccagcaacagctgttaagccagggtttgcctgccttgatcgagcaggattttgccacgttcagccgttttcttggtgatttgcaagcttataatgcagattatttcgcgcctgcacagggtggtgcctatgccagttcgagcgtggcaagcattttgcaatctattaaaaaacaaggttatgcaggcatcggacagacgtcctggggaccaacaggatttgtgttgctgccttcgcgtgcagaagcggtcactatgcaaatgcagctgctgcatttgcatgctaacgatgcctccctgggatttatcgtcacagcagccatgaatcagtcggccaatattatgtttgggaatggcgcagattaaattttcttaagataattttgaaaagttatggcttttacggtctactctttattttgaactggtctggatatgtgtatattggcgaaagatattgttaacgaaccgtaccggggggaagaatgaaaaaaaccagtattatgcatttgttcactgctgccaagaatgccagtccatttgatgtgaatatggcctttgatgctggctatgagaaaattatttcttacaccgatgtgactttgaatgaaatcgtcgcgttgacgcaggatgccattttttcacgcagcccgagtggattaaagcagcaagccttattttttggtggccgcgatatccaggtggcgctggaaatgcagaagcaggcgcgcagtgccatgttcaagccatttgaatgccatactttttctgatccgtccggtgcctttaccacggcagcagccatgctggccaaagtcgatttttatttgcagaaatctggtagtggtttgggcaaggaaaaagtcgctatttttggtgccagtggtaccgtgggctcgacagcagcactcatcgcagctcgccagggagccactgtattgatggtggcgcactcggatgttgccagtatgcaggcgtatgttgataagctttctagcaattatgatgtcagcctcaaagtagtggatggcagtacagaggctgccaaagtggctgtgttgaatgaagcgacagtagccttgtgtgcaacaccagctgggattcgcgtccttgaaatcaagcaattcgccaactccaaatcactgaaagtggtggcagacgtaaacgcagtccctccttctggcattgagggcgtagacacattctctgatggtggcgtgattgaaggcacacaagtggccggttttggcgccttggcgattggccagttgaaatatgtcacccaaaacaagctactggagcaaatgctgcaaagcgaaagccccatgcacattgattaccatgaggcatatgagtatgcctgtgcacacgtggagtaaagcgattcttgcgattggctgtattgtcacagagtgcgcgtatttatagccagatggcgcaacaagaaggctttagtgtattggctgtggacgcgtttgcggataacgatacgcagcaatctgcaacattggtataccactggccaggcctgtgcggaccggatgtcaacaatgaaatgtctgggttaatggaagtattggatagtttcaagccggatgccgttttacttggttctggttttgaagcagatcaagcggcatatgcaaagttattcacacgccatgcaatatttggcaatacaccggaaaccgtggcccgggtcaaaaatccgcaatggctgaaaaattattgtgatgcgcacggcgtccagtcgccatgcatcgccacgcaaaagccggtcgaaggtcgttggctgcataaacaggcgggacgatgtggtggtatgcatgtgcaagactggtcacctgcagcaacagtcactgcaaaaagttactggcaagcatttcagccaggacaagccgtgggaatattgtttgtcgcgcatcagcaggcattcacattgattggcgtacatgcactcaagcaacgcgcagggagctatgcttatgcaggcgtgaagcgcttgcatgatccagcgctaactgtcgctgccacagagttattgcaggcagtcttgccaggcttgggattagttggcattaacagtattgatgccatctggcatgagggcgagttgcatcgtcgacaggt

SEQ ID NO: 22 - protein, 297 aa, Methylophilus methylotrophus
MKKTSIMHLFTAAKNASPFDVNMAFDAGYEKIISYTDVTLNEIVALTQDAIFSRSPSGLKQQALFFGGRDIQVALEMQKQARSAMFKPFECHTFSDPSGAFTTAAAMLAKVDFYLQKSGSGLGKEKVAIFGASGTVGSTAALIAARQGATVLMVAHSDVASMQAYVDKLSSNYDVSLKVVDGSTEAAKVAVLNEATVALCATPAGIRVLEIKQFANSKSLKVVADVNAVPPSGIEGVDTFSDGGVIEGTQVAGFGALAIGQLKYVTQNKLLEQMLQSESPMHIDYHEAYEYACAHVE

SEQ ID NO: 23 - DNA, 1823 bp, Methylophilus methylotrophus
  CDS (522)..(1496)
ggcaggatccttgattggcgtacatgcactcaagcaacgcgcagggagctatgcttatgcaggcgtgaagcgcttgcatgatccagcgctaactgtcgctgccacagagttattgcaggcagtcttgccaggcttgggattagttggcattaacagtattgatgccatctggcatgagggcgagttgcatctcatcgaggtgaacccccgactcagcgccagtatgcgtctgtatgcagggttgccattgataaaagcgcatatggacagttgcaatggcaacatcatgcctctgcaacaacatactaaaacgcatgcctgccattgcattgcgtatgcacgacaagagattaacgcaagtcatctagactttcctgactggttggaagaccggcccagtggtggcatgattgctgcgggtctgcccgtttgcagtctatatgcacaaggggactcagacagggaattgctacaggctttgcaagataagaaaacacgattagagaaactatgggggacttatgtctgtaaccgcatcgaattcaacatccattagcgttcaacaatatagcgcaccactggtggcgcatctgatggccaatgccccagctttaggctgcgcagtggcaacgcatgaaacaggcgccacgattgtggatgcaggtattcaagcaactggcggcctggaagcagggcgcatcatcgccgaaatttgcatgggtggtttaggtagagtgtcgttgcagcaagtgccgcaatttgcccactggcctctcagtgtcgtggtgacagctacccaaccggtgattgcctgccttggcagtcagtatgccggctgggccttgtcacacgaaaaattcttctcactgggcagtggcccggcacgctcaattgcacagcgtgaagaagtcttcaaagatattaattacagtgataaaggcgagcaaacggttttggtgctggaaaccgacaaggtgcctcctgtgcaggtgattgaaaaagtggccagagatactggcctgccagccaataagctgacatttatcctgaccccaacccgcagtgtggccggttccttgcaagtgactgcacgtgtgctcgaagttgcactgcataaatgccatgccttgcattttgacctgaatgccattgtcgatggttatggtgtcgcgccagtaccggcgccctcgccagactttatcgtcggcatgggccgtaccaatgatgcgatcctgtttggcggctttgtgcagttgtttgtgaataccgatgatgctgcagcggaacaactcgcccagcaactaccttcctcttcatccaaagattacggccgcccattcgcacaggtgttcaaagccgttaatatggacttttaccagattgaccccatgttgttctctccagccaaagtcagtgtgactaacctcaagtccggcaagactttctttggcggccagtttaatgaaacccttctgaatcaatcatttggaagttaaatttaaggtgctataaaagttcttgacgcgggattctgtgcaaaatgcatgggtcccgcgtgatcatttcaacgctgacatgaacgtccttcctatttttaccgacgaagtagcccaggctggtggctggcacggacagagtctggcgcaagcctttgcaaaacttggctggcaggcattgatggtgtcattggatagttgccacgtcagtattgtgaatcagcaggtgcaagtccacatcccagggctcacacaagctgcacctttggcattcgtgcgtggcgtggcggcgggcaccacccagcaaatcatcacgcgtcgacatat

SEQ ID NO: 24 - protein, 324 aa, Methylophilus methylotrophus
MSVTASNSTSISVQQYSAPLVAHLMANAPALGCAVATHETGATIVDAGIQATGGLEAGRIIAEICMGGLGRVSLQQVPQFAHWPLSVVVTATQPVIACLGSQYAGWALSHEKFFSLGSGPARSIAQREEVFKDINYSDKGEQTVLVLETDKVPPVQVIEKVARDTGLPANKLTFILTPTRSVAGSLQVTARVLEVALHKCHALHFDLNAIVDGYGVAPVPAPSPDFIVGMGRTNDAILFGGFVQLFVNTDDAAAEQLAQQLPSSSSKDYGRPFAQVFKAVNMDFYQIDPMLFSPAKVSVTNLKSGKTFFGGQFNETLLNQSFGS

(19) United States
(12) Patent Application Publication
(10) Pub. No.: US 2004/0171134 A1
(43) Pub. Date: Sep. 2, 2004

(54) Method for producing recombinant of methanol-assimilating bacterium

(76) Inventors: Takayuki Asahara, Kawasaki-shi (JP); Hisashi Yasueda, Kawasaki-shi (JP)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/751,928

(22) Filed: Jan. 7, 2004

(30) Foreign Application Priority Data
Jan. 8, 2003 (JP) 2003-1927

(51) Int. Cl. C12N 1/20

(52) U.S. Cl. 435/252.3

(57) ABSTRACT

A recombinant of a methanol-assimilating bacterium in which an exogenous linear DNA fragment is introduced into its chromosomal DNA, and is prepared by the following steps:

(a) preparing an exogenous linear DNA fragment comprising a nucleotide sequence identical to a nucleotide sequence of an arbitrary region of said chromosomal DNA,

(b) introducing the linear DNA fragment into the methanol-assimilating bacterium to obtain recombinants, and

(c) selecting a recombinant in which said region on the chromosome is replaced with said linear DNA fragment.

### BACKGROUND OF THE INVENTION

1. Field of the Invention

The present invention relates to a method for producing a recombinant of a methanol-assimilating bacterium. More precisely, the present invention relates to a method for producing a recombinant whereby a gene on a chromosome is replaced with an exogenous gene. The present invention is useful in methods of breeding or improving methanol-assimilating bacteria.

2. Description of the Related Art

In order to delete, amplify or modify a desired gene on a chromosome of a methanol-assimilating bacterium by gene substitution, typically a method of incorporating a recombinant plasmid which is capable of conjugative transfer and which carries a DNA segment containing the desired gene into a plasmid DNA donor bacterium is utilized. This method enables conjugative transfer of the recombinant plasmid to a methanol-assimilating bacterium. Alternatively, a method of introducing a plasmid DNA having a DNA segment containing a desired gene into a methanol-assimilating bacterium by electroporation and causing homologous recombination between the desired gene on a chromosome and the DNA segment on the introduced plasmid may be utilized. Examples thereof which have been disclosed to date include, for example, disruption of an desired gene in a Methylobacterium extorquens strain having the serine pathway or a Methylobacillus flagellatus strain having the ribulose monophosphate pathway (J. Bacteriol., vol. 176, pp.4052-4065 (1994), Microbiology, vol. 146, pp.233-238 (2000)).

Methods described above use circular DNAs and are useful in gene substitution for many procaryotes (Nature, vol. 289, pp.85-88 (1981)), however, it is believed that methods for substitution of a desired gene using linear DNAs, which will be described herein, are inapplicable to most procaryotes (Proc. Natl. Acad. Sci. USA, vol. 97, pp.6640-6645 (2000)).

The gene substitution technique using linear DNA has been exclusively used for yeast, fungi, Bacillus subtilis, and the like. This method is extremely simple and advantageous in that it does not require a series of time-consuming operations, as is required in methods using circular DNA, i.e., the first homologous recombination reaction of a circular DNA and a homologous region on a chromosome, second homologous recombination reaction and selection of a recombinant in which the desired gene as a target is replaced from a group of obtained recombinants. Furthermore, in many cases, the desired gene substitution does not occur in the recombinants obtained by the second homologous recombination reaction, and they return to the gene structure before the operations, which results in the pain-staking selection of a strain having the desired gene structure occurring at a low frequency from many recombinants.

However, in Escherichia coli, the methods for substitution of a desired gene using a circular DNA have constituted the mainstream methods. It is said that this is because Escherichia coli has a powerful enzymatic activity for degrading introduced linear DNA. Therefore, in Escherichia coli, gene substitution, deletion and modification using a linear DNA have been possible only in strains in which the enzymatic activity is reduced (Marinus M.G., et al., Mol. Gen. Genet., 192, pp 288-289 (1983), Russell C. B., et al., J. Bacteriol., 171, pp.2609-2613 (1989)).

Furthermore, in methanol-assimilating bacteria, only gene substitution methods using a circular DNA have been known to date.

### SUMMARY OF THE INVENTION

An object of the present invention is to provide a simple gene substitution method for breeding and improvement of methanol-assimilating bacteria.

It is a further object of the present invention to provide a method for producing a recombinant of a methanol-assimilating bacterium in which a exogenous linear DNA fragment is introduced into the chromosomal DNA of the methanol-assimilating bacterium comprising:

(a) preparing an exogenous linear DNA fragment comprising a nucleotide sequence identical to a nucleotide sequence of an arbitrary region of said chromosomal DNA,

(b) introducing said linear DNA fragment into the methanol-assimilating bacterium to obtain recombinants, and

(c) selecting a recombinant in which said region on the chromosome is replaced with said linear DNA fragment.

It is a further object of the present invention to provide a method as described above, wherein said methanol-assimilating bacterium is a Methylophilus bacterium.

It is a further object of the present invention to provide a method as described above wherein said methanol-assimilating bacterium is Methylophilus methylotrophus.

It is a further object of the present invention to provide a method as described above, wherein said linear DNA fragment comprises a segment having said nucleotide sequence identical to the arbitrary region of said chromosomal DNA, and another sequence inserted into the segment.

It is a further object of the present invention to provide a method as described above, wherein said linear DNA fragment comprises partial deletion or substitution of one or more nucleotides.

According to the present invention, transformation, especially gene substitution, of methanol-assimilating bacteria can be efficiently performed in a simple manner.

### DESCRIPTION OF THE PREFERRED EMBODIMENTS

The inventors of the present invention considered that if gene substitution utilizing an exogenous linear DNA can be carried out in methanol-assimilating bacteria, genetic manipulation, including chromosomal manipulation, should be possible in the breeding of methanol-assimilating bacteria for industrial use, and thereby time and cost are significantly saved. Thus, the inventors assiduously studied in order to achieve the aforementioned objects. As a result, they found that, in Methylophilus bacteria, a recombination reaction efficiently occurred between DNAs having the same sequences even when they were linear DNAs, and therefore substitution, deletion and modification of a desired gene could be possible. Thus, they accomplished the present invention.

Hereafter, the present invention will be explained in detail.

The present invention provides a method for producing a recombinant of a methanol-assimilating bacterium. The present invention also provides a method for transformation of a methanol-assimilating bacterium, or a method for gene substitution in a methanol-assimilating bacterium.

The methanol-assimilating bacterium in the present invention is a bacterium capable of utilizing methanol as a carbon source, and a strict methanol-assimilating bacterium is preferred. Examples of strict methanol-assimilating bacterium include, for example, Methylophilus bacteria, which are gram-negative bacilli, such as Methylophilus methylotrophus, Methylobacillus bacteria such as Methylobacillus glycogenes and Methylobacillus flagellatum, and so forth. Specific examples include the Methylophilus methylotrophus AS1 strain (NCIMB 10515), Methylobacillus glycogenes NCIMB 11375 strain, ATCC 21276 strain, ATCC 21371 strain, ATR80 strain, A513 strain (described in Appl. Microbiol. Biotechnol., vol. 42, pp.67-72 (1994)), Methylobacillusflagellatum KT strain (described in Arch. Microbiol., vol. 149, pp.441-446 (1988)) and so forth. Among these, the NCIMB 10515 strain and NCIMB 11375 strain can be obtained from National Collections of Industrial and Marine Bacteria (Address: NCIMB Lts., Torry Research Station 135, Abbey Road, Aberdeen AB9 8DG, United Kingdom).

In the present invention, the “exogenous linear DNA fragment” means a DNA fragment which is to be introduced into the methanol-assimilating bacterium from the outside of the bacterium. The origin of the exogenous linear DNA fragment may be an organism other than the methanol-assimilating bacterium host, or may be the methanol-assimilating bacterium host itself, or a bacterium of the same species.

In the present invention, the “recombinant” means a methanol-assimilating bacterium in which the linear DNA fragment is introduced into its chromosomal DNA by homologous recombination. When the linear DNA fragment contains one or more nucleotide substitutions, the recombinant obtained by the method of the present invention may not be structurally distinguished from a mutant strain obtained by a mutagenesis treatment. However, such a recombinant is included in the “recombinant” as long as it is obtained by the method of the present invention.

The linear DNA used in the present invention is a linear DNA fragment comprising a nucleotide sequence identical to a nucleotide sequence of an arbitrary region of a chromosomal DNA (henceforth also referred to as a “target region”).

The “linear DNA fragment” means a DNA fragment having free 5′ end and 3′ end, and means that it is not a “circular DNA.” Furthermore, the actual form of the linear DNA fragment may not necessarily be linear, and it may have a bend or torsion. Although the linear DNA fragment may be double-stranded or single stranded when it is introduced into a Methylophilus bacterium, it is preferably double-stranded.

One embodiment of the linear DNA fragment used in the present invention includes, for example, a segment having a nucleotide sequence identical to that of an arbitrary region of a chromosomal DNA, as well as another sequence inserted into the segment. Examples of other sequences include the marker gene described later. Furthermore, in another embodiment, the linear DNA fragment has a nucleotide sequence identical to that of a target region, but includes a partial deletion or substitution of one or more nucleotides. In this embodiment, the portion other than the portion of the aforementioned deletion or nucleotide substitution of the linear DNA fragment preferably has the same nucleotide sequence as that of the target region.

The segment having the same nucleotide sequence as that of the target region can be obtained by cloning the target region on the chromosome of the methanol-assimilating bacterium. The target region may be, for example, cloned into a plasmid from the chromosomal DNA to obtain a recombinant plasmid, and then excised from the recombinant plasmid with a restriction enzyme, or it may be obtained by directly amplifying the desired fragment from the genomic DNA by PCR.

When such a linear DNA fragment as described above (henceforth also referred to as the “DNA fragment for introduction”) is introduced into a methanol-assimilating bacterium host, and a homologous recombination reaction occurs between a sequence on the host chromosomal DNA which is identical with at least a part of the DNA fragment for introduction (henceforth referred to as the “target region”) and the DNA fragment for introduction, insertion of the DNA fragment for introduction and removing of the target region occurs simultaneously. As a result, the target region is replaced by the DNA fragment for introduction.

When the aforementioned target region is a gene (henceforth referred to as a “target gene”), and the DNA fragment to be introduced is a gene having identity with the aforementioned gene but having a partially different sequence (henceforth referred to as a “gene for introduction”), the target gene is replaced with the gene for introduction (henceforth referred to as “gene substitution”). The gene for introduction may be a fusion gene consisting of two or more of genes or a gene complex containing two or more of genes.

When the aforementioned target region is a structural gene coding for a protein, and the gene for introduction does not code for any protein having an activity due to deletion of a partial sequence or insertion of another sequence (henceforth referred to a “disrupted-type gene”), the target gene is disrupted by the gene substitution. Furthermore, by modifying a nucleotide sequence of an expression regulatory sequence of the target gene, expression of the target gene can be reduced.

Furthermore, if a mutant gene having one or more arbitrary mutations is used as the gene for introduction, the arbitrary mutations can be introduced into the target gene by the gene substitution. This “introduction of mutations” includes replacement of mutations of the target gene contained in the methanol-assimilating bacterium with wild-type or other mutations.

The deletion, insertion or mutation in the aforementioned gene for introduction may concern either one nucleotide or a region consisting of two or more nucleotides. Such modifications of the gene for introduction can be performed by the site-specific substitution method.

In order to determine whether the substitution of the DNA fragment for introduction by the target region has occurred as intended, for example, a drug resistance marker gene having resistance to an antibiotic may be incorporated into the DNA fragment for introduction. However, such a marker gene needs to have sequences on either side that is identical to the target region. Drug resistance marker genes include, but are not limited to a gene imparting resistance to a drug such as kanamycin, gentamycin, tetracycline, ampicillin or streptomycin. Such a marker gene as described above can be used for construction of a disrupted-type gene by inserting it into the gene for introduction.

The disrupted-type gene inserted with a marker gene may be prepared by a gene recombination technique using a plasmid DNA as shown in the examples section, or it can be prepared by simultaneously performing amplification of the gene for introduction and insertion of the marker gene by crossover PCR.

When a strain in which the desired gene on a chromosome is replaced with the DNA fragment for introduction can be selected according to a phenotype or genotype, it is not necessarily required to use a drug resistance marker gene. A genotype may be easily confirmed by, for example, hybridization or PCR.

Furthermore, the length of the DNA segment identical to the target region in the DNA fragment for introduction needs to be of such a length that the homologous recombination can occur in the methanol-assimilating bacterium. Specifically, it is usually a length of 20 or more nucleotides, preferably 500 or more nucleotides, more preferably 1000 or more nucleotides. Such a DNA fragment can be recognized by an enzyme for homologous recombination in a host cell, and homologous recombination proceeds between the DNA fragment for introduction and the target region on a chromosome. The DNA fragment for introduction may include a DNA segment which is not identical to the target region in a region upstream and/or downstream from the DNA region identical to the target region.

When the gene for introduction contains a mutation or insertion, each of upstream and downstream regions of the mutation or insertion site preferably has a length of 500 or more nucleotides, and it is a length of around 5000 nucleotides at most. Furthermore, when a drug resistance marker gene is inserted into the gene for introduction, each of the segments identical to the target region at regions upstream and downstream from the marker gene preferably has a length of 500 or more nucleotides.

Furthermore, according to the gene substitution method of the present invention, it is also possible to introduce a gene that is not inherently contained in a methanol-assimilating bacterium host into an unnecessary gene on a chromosome. The DNA fragments having sequences identical to upstream and downstream regions of the unnecessary gene on a chromosome are ligated to the both ends of DNA containing the gene for introduction to prepare a linear DNA fragment, and the resulting linear DNA fragment is used to transform a methanol-assimilating bacterium. The DNA fragment introduced as described above causes a recombination reaction with genes on a chromosome identical to the nucleotide sequences of the both ends of the DNA fragment, thereby the unnecessary gene is deleted, and instead, a foreign gene is inserted at the site of the deletion.

As for the methods of digestion, ligation and hybridization of DNA, PCR and so forth, usual methods well known to those skilled in the art can be used. Such methods are described in Sambrook, J., Fritsch, E. F., and Maniatis, T., “Molecular Cloning A Laboratory Manual, Second Edition”, Cold Spring Harbor Laboratory Press (1989) and so forth.

Examples of the method for introducing the exogenous linear DNA fragment into a methanol-assimilating bacterium include, but are not limited to electroporation (described in Canadian Journal of Microbiology, 43, 197 (1997)) and so forth. For example, the exogenous linear DNA fragment can be introduced into a methanol-assimilating bacterium using a commercially available apparatus (GenePulser produced by BioRad etc.) according to a method defined for the apparatus.

A strain in which substitution of a target gene on a chromosome has occurred can be selected based on a phenotype of the target gene, gene for introduction or marker gene, or a genotype of each gene.

For example, in the case of Methylophilus methylotrophus, the SEII medium described in the following examples can be used as a base medium used for evaluation of drug resistance. By adding an appropriate drug to the base medium and culturing the bacterium at an appropriate temperature in the range of 20 to 40° C. for about 12 to 100 hours in the medium to select a strain resistant to the drug, a strain in which a desired gene is replaced with an introduced DNA fragment can be obtained.

### EXAMPLES

Hereinafter, the present invention is explained more specifically, with reference to the following non-limiting examples.

### Example 1

### Method for Disrupting RecA Gene Using Linear DNA

A recA gene-deficient strain was constructed from a wild-type strain of Methylophilus methylotrophus, the AS1 strain (NCIMB No. 10515), using a linear DNA as follows.

The AS1 strain was inoculated into 50 mL of the SEII medium (composition: 1.9 g/L of K₂HPO₄, 5.0 g/L of (NH₄)₂SO₄, 1.56 g/L of NaH₂PO₄.2H₂O, 0.2 g/L of MgSO₄.7H₂O, 0.72 mg/L of CaCl₂.6H₂O, 5 μg/L of CuSO₄.5H₂O, 25 μg/L of MnSO₄.5H₂O, 23 μg/L of ZnSO₄.7H₂O, 9.7 mg/L of FeCl₃.6H₂O, 1% (v/v) of CH₃OH) and cultured overnight at 37° C. Then, the culture broth was centrifuged to collect the cells. Chromosomal DNA was purified from the obtained cells using a commercially available kit (Genomic DNA Purification Kit (produced by Edge Biosystems)).

PCR was performed using the chromosomal DNA obtained as described above as a template and the primer DNAs (mRecA-F3, mRecA-R3) shown in SEQ ID NOS: 1 and 2. As for the reaction condition, a cycle of denaturation at 94° C. for 10 seconds, annealing at 55° C. for 30 seconds and extension reaction at 70° C. for 2 minutes was repeated for 28 cycles. In addition, a commercially available kit (Pyrobest Taq (produced by Takara Bio Inc.)) was used as a heat-resistant DNA polymerase.

A DNA fragment of about 1.3 kilobase pairs (“kbp”) containing the recA gene was obtained by PCR as described above. The nucleotide sequence of this DNA fragment is shown in SEQ ID NO: 19, and the encoded amino acid sequence is shown in SEQ ID NO: 20. Both ends of this DNA fragment were blunt-ended and phosphorylated using BKL Kit (Takara Bio Inc.). The plasmid pUCl9 (Takara Bio Inc.) was treated with the restriction enzyme BamHI and then similarly blunt-ended, and the 5′ phosphate of the digested ends were dephosphorylated.

The above two DNA fragments were ligated using Ligation Kit (Takara Bio Inc.) to construct pUC-MrecA 1. The direction of the recA gene in this plasmid was the same as the direction of transcription from the lac promoter in the plasmid.

Then, pUC-MrecAl was digested with the restriction enzyme BamHIl, and the digested ends were further dephosphorylated to prepare a DNA fragment in which the recA gene was split. Furthermore, the plasmid pUC4K (produced by Amersham Biosciences) was treated with the restriction enzyme BamHI to prepare a DNA fragment (1.3 kbp) containing the kanamycin resistance gene (Km^{R}). Both DNA fragments mentioned above were ligated using Ligation Kit to obtain pUC-MrecA1::km.

The plasmid pUC-MrecA1::km was digested with the restriction enzymes XbaI and KpnI, and the digested product was subjected to electrophoresis to purify and obtain a recA gene DNA fragment inserted with the kanamycin resistance gene (recA::Km^{R}). This DNA fragment was concentrated and desalted by ethanol precipitation.

The Methylophilus methylotrophus AS1 strain was cultured at 37° C. for 16 hours with shaking in the SEII liquid medium (methanol concentration: 0.5% (v/v)), and 20 mL of the culture broth was centrifuged at 10,000 rpm for 10 minutes to collect the cells. 1 mM HEPES buffer (pH 7.2, 20 ml) was added to the cells to suspend the cells in the buffer, and the suspension was centrifuged. This operation was repeated twice, and 1 ml of the same buffer was finally added to the cells to prepare a cell suspension as electro cells for electroporation.

About 1 μg of the aforementioned recA gene DNA fragment was added to 100 μL of the electro cells, and an electric pulse was applied to the cells at 18.5 kV/cm, 25 μF and 200 Ω to perform the electroporation. This cell suspension was immediately added to the SEII liquid medium and cultured at 37° C. for 3 hours. Then, this culture broth was applied to a SEII +Km agar medium (SEII medium containing 20 μg/ml of kanamycin and 1.5% (w/v) of agar), and the cells were cultured at 37° C. for three days. As a result, fifty transformants of kanamycin resistant were obtained. Furthermore, seven strains among them were spread again on the SEII +Km agar medium to further purify the colonies. Chromosomal DNA was extracted from one colony, and the structure of the recA gene was analyzed by PCR. The DNA primers were mRecA-F2, mRecA-R2, Km4-F1 and Km4-R1 (having the sequences of SEQ ID NOS: 3, 4, 5 and 6, respectively). First, when PCR was performed using mRecA-F2 and Km4-R1 as a pair of DNA primers and the genomic DNA of the candidate strain as a template, it was confirmed that a DNA fragment having a size of 1530 bp was amplified (the reaction conditions were 94° C. for 10 seconds for denaturation, 50C for 30 seconds for annealing reaction and 72° C. for 2 minutes for extension reaction). Furthermore, when Km4-F1 and mRecA-R2 were used, a DNA fragment having a size of 1950 bp was amplified. This indicated that the recA gene region on the genome of the candidate strain was disrupted by the KM^{R} gene.

Furthermore, the phenotype of the aforementioned candidate strain with disruption of the recA gene was comfirmed. That is, the recA gene product is an enzyme involved in homologous recombination of DNA and also is involved in the SOS repair mechanism of the cells. Therefore, if the recA gene of the strain was disrupted, and thus the recA function eliminated, the strain would show high sensitivity to ultraviolet irradiation (UV). Therefore, the UV sensitivity of the candidate strain was compared with that of the wild-type strain AS1.

A candidate for recA-disrupted strain and the AS1 strain were each cultured in 3 mL of the SEII medium for 16 hours, and a part of the culture broth, 300 μL, was inoculated to 3 mL of the same medium. Subsequently, the cells were cultured at 37° C. until the cells reached the logarithmic phase (OD is about 0.5). Then, this culture broth was serially diluted with the SEII liquid medium to prepare dilutions diluted to a degree of 106 times from the original culture broth (1-fold dilution). Furthermore, 15 μL of each dilution was spotted to a surface of the SEII agar medium plate, left for a while to allow each cell suspension to infiltrate into the agar, and dried. Then, the agar plate spotted with the cell suspension was placed under a UV light (Toshiba germicidal lamp, GL15) at a distance of 80 cm and irradiated with a UV ray for 15 seconds. Separately, control plots were also prepared in which UV irradiation was not performed. Both of these agar plates were incubated at 37° C. for one day, and the formation of colonies of each bacterium produced from the spotting site was observed. As a result, it was found that, as expected, the candidate strain of the recA disrupted strain showed UV sensitivity about 1000 times higher than that of the AS1 strain, and thus the obtained kanamycin resistant strain was a recA-disrupted strain also by phenotype.

Thus, disruption of the recA gene in Methylophilus methylotrophus using a linear DNA was confirmed.

### Example 2

### Method for Disrupting MtdA Gene (Methylene Tetrahydromethanopterin Tetrahydrofolic Acid Dehydrogenase Gene) Using Linear DNA

Chromosomal DNA was prepared from the AS1 strain in the same manner as in Example 1. The chromosomal DNA was used as a template, and MmtdA-F 1 and MmtdA-R1 (SEQ ID NOS: 11 and 12, respectively) were used as DNA primers to perform PCR using a commercially available kit, Pyrobest Taq (produced by Takara Bio Inc.) (reaction conditions: denaturation at 94° C. for 10 seconds, annealing at 50° C. for 30 second, and extension reaction at 70° C. for 3 minutes). As a result, a DNA fragment of the mtdA gene having a length of about 2.1 kbp was amplified. The nucleotide sequence of this DNA fragment is shown in SEQ ID NO: 21, and the amino acid sequence encoded thereby is shown in SEQ ID NO: 22. Subsequently, this DNA fragment was digested with the restriction enzymes BamHI and Sall and then purified.

Separately, a general-purpose plasmid vector, pBluescriptIlSK-(Stratagene), was similarly digested with BamHI and Sall. This vector fragment and the aforementioned mtda gene fragment were ligated using Ligation Kit to construct pBS-MmtdAl. Then, this plasmid was digested with the restriction enzymes EcoRV and MAul to prepare a DNA fragment in which the mtdA gene region was split.

Furthermore, the DNA primers for PCR, Km4-F2 and Km4-R2 (shown in SEQ ID NOS: 7 and 8, respectively) were produced and PCR was performed using these primes and pUC4K2 as a template (conditions: denaturation at 94° C. 10 seconds, annealing at 50° C. for 30 seconds, and extension reaction at 70° C. for 1.5 minutes) to amplify a DNA fragment carrying the Km^{R} (kanamycin resistance) gene. Furthermore, the both ends of this DNA fragment were digested with EcoRV and Mlul, and the DNA fragment was purified.

The two aforementioned fragments were ligated using Ligation Kit to construct pBS-MmtdA1Δ, and the resulting plasmid pBS-MmtdA1 Δ was digested with the restriction enzymes BamHI and SalI to prepare a mtdA::Km^{R} gene fragment consisting of the mtdA gene in which the Km^{R} gene was inserted. This digestion product was concentrated by ethanol precipitation and further subjected to a desalting treatment, and the resultant was used as a DNA sample for electroporation.

In the same manner as in Example 1, the aforementioned DNA sample was introduced into the AS1 strain by electroporation to obtain about 50 strains of transformants as Km^{R} strains. Six strains were selected from these, and the genomic DNA of each candidate strain was used as a template to perform PCR (conditions: denaturation at 94° C. 10 seconds, annealing at 50° C. for 30 second, and extension reaction at 72° C. for 2.5 minutes) to examine the structure of the mtdA gene region of each candidate strain. The DNA primers used for PCR for this assay were MmtdA-F2, MmtdA-R2, Km4-F1 and Km4-R1 (SEQ ID NOS: 9, 10, 5 and 6, respectively). As a result, a DNA fragment having a size of 2 kbp and a DNA fragment having a size of 1.6 kbp were amplified with the combination of MmtdA-F2 and Km4-R1 and the combination of MmtdA-R2 and Km4-F 1, respectively, as expected, and thus the deficiency of the mtdA gene, which was the target gene of the disruption, was confirmed.

### Example 3

### Disruption of Mch Gene (Methenyltetrahydromethanopterin Cyclohydrolase Gene) Using Linear DNA

Chromosomal DNA was prepared from the AS1 strain in the same manner as in Example 1. This DNA was used as a template, and Mmch-F1 and Mmch-R1 (SEQ ID NOS: 13 and 14, respectively) were used as DNA primers to perform PCR (reaction conditions: denaturation at 94° C. for 10 seconds, annealing at 50° C. for 30 second, and extension reaction at 70° C. for 2 minutes). As a result, a DNA fragment of the mch gene having a length of about 1.8 kbp was amplified. The nucleotide sequence of this DNA fragment is shown in SEQ ID NO: 23, and the amino acid sequence encoded thereby is shown in SEQ ID NO: 24. Subsequently, this DNA fragment was digested with the restriction enzymes BamHI and SalI and then purified.

Separately, a general-purpose plasmid vector, pBluescriptIISK-(Stratagene), was similarly digested with BamHI and SalI. This vector fragment and the aforementioned mch gene fragment were ligated using Ligation Kit to construct pBS-Mmch1. Then, the obtained plasmid was digested with the restriction enzymes EcoRI and PstI to prepare a DNA fragment in which the mch gene region was split.

Furthermore, the DNA primers for PCR, Km4-F3 and Km4-R3 (shown in SEQ ID NOS: 15 and 16, respectively) were produced and PCR was performed using the primers and pUC4K2 as a template (conditions: denaturation at 94° C. 10 seconds, annealing at 50° C. for 30 seconds, and extension reaction at 70° C. for 1.5 minutes) to amplify a DNA fragment carrying Km^{R} (kanamycin resistance) gene. Furthermore, the both ends of this DNA fragment were digested with EcoRI and PstI, and the DNA fragment was purified.

The aforementioned two fragments were ligated using Ligation Kit to construct pBS-Mmch1 Δ, and the plasmid pBS-Mmch1 Δ was digested with the restriction enzymes BamHI and SalI to prepare a mch::Km^{R} gene fragment consisting of the mch gene in which the Km^{R} gene was inserted. This digestion product was concentrated by ethanol precipitation and further subjected to a desalting treatment, and the resultant was used as a DNA sample for electroporation.

In the same manner as in Example 1, the aforementioned DNA sample was introduced into the AS1 strain by electroporation to obtain about 50 strains of transformants as Km^{R} strains. Six strains were selected from them, and the genomic DNA of each candidate strain was used as a template to perform PCR (conditions: denaturation at 94° C. for 10 seconds, annealing at 50° C. for 30 second, and extension reaction at 72° C. for 1.5 minutes) to examine the structure of the mch gene region of each strain. The DNA primers used for PCR were Mmch-F2, Mmch-R2, Km4-F1 and Km4-R1 (SEQ ID NOS: 17, 18, 5 and 6, respectively). As a result, amplification of a DNA fragment having a size of 1.8 kbp and a DNA fragment having a size of 2.5 kbp was confirmed with the combination of Mmch-F2 and Km4-R1 and the combination of Mmch-R2 and Km4-F1, respectively, as expected, and thus it was confirmed that strains deficient in mch, which was the target gene of the disruption, were prepared.

While the invention has been described in detail with reference to preferred embodiments thereof, it will be apparent to one skilled in the art that various changes can be made, and equivalents employed, without departing from the scope of the invention. Each of the aforementioned documents, including the foreign priority document JP2003-1927, is incorporated by reference herein in its entirety.

We claim:

1. A method for producing a recombinant of a methanol-assimilating bacterium in which an exogenous linear DNA fragment is introduced into the chromosomal DNA of the methanol-assimilating bacterium comprising:
(a) preparing an exogenous linear DNA fragment comprising a nucleotide sequence identical to a nucleotide sequence of an arbitrary region of said chromosomal DNA,
(b) introducing said linear DNA fragment into the methanol-assimilating bacterium to obtain recombinants, and
(c) selecting a recombinant in which said region on the chromosome is replaced with said linear DNA fragment.

2. The method according to claim 1, wherein said methanol-assimilating bacterium is a Methylophilus bacterium.

3. The method according to claim 1, wherein said methanol-assimilating bacterium is Methylophilus methylotrophus.

4. The method according to claim 1, wherein said linear DNA fragment comprises a segment having said nucleotide sequence identical to the arbitrary region of said chromosomal DNA, and another sequence inserted into the segment.

5. The method according to claim 1, wherein said linear DNA fragment comprises partial deletion or substitution of one or more nucleotides.
